SEQUENCE LISTING (9 sequences)

SEQ ID NO: 1 - DNA, 2418 bp, Homo sapiens
atgtcaagctcttcctggctccttctcagccttgttgctgtaactgctgctcagtccaccattgaggaacaggccaagacatttttggacaagtttaaccacgaagccgaagacctgttctatcaaagttcacttgcttcttggaattataacaccaatattactgaagagaatgtccaaaacatgaataatgctggggacaaatggtctgcctttttaaaggaacagtccacacttgcccaaatgtatccactacaagaaattcagaatctcacagtcaagcttcagctgcaggctcttcagcaaaatgggtcttcagtgctctcagaagacaagagcaaacggttgaacacaattctaaatacaatgagcaccatctacagtactggaaaagtttgtaacccagataatccacaagaatgcttattacttgaaccaggtttgaatgaaataatggcaaacagtttagactacaatgagaggctctgggcttgggaaagctggagatctgaggtcggcaagcagctgaggccattatatgaagagtatgtggtcttgaaaaatgagatggcaagagcaaatcattatgaggactatggggattattggagaggagactatgaagtaaatggggtagatggctatgactacagccgcggccagttgattgaagatgtggaacatacctttgaagagattaaaccattatatgaacatcttcatgcctatgtgagggcaaagttgatgaatgcctatccttcctatatcagtccaattggatgcctccctgctcatttgcttggtgatatgtggggtagattttggacaaatctgtactctttgacagttccctttggacagaaaccaaacatagatgttactgatgcaatggtggaccaggcctgggatgcacagagaatattcaaggaggccgagaagttctttgtatctgttggtcttcctaatatgactcaaggattctgggaaaattccatgctaacggacccaggaaatgttcagaaagcagtctgccatcccacagcttgggacctggggaagggcgacttcaggatccttatgtgcacaaaggtgacaatggacgacttcctgacagctcatcatgagatggggcatatccagtatgatatggcatatgctgcacaaccttttctgctaagaaatggagctaatgaaggattccatgaagctgttggggaaatcatgtcactttctgcagccacacctaagcatttaaaatccattggtcttctgtcacccgattttcaagaagacaatgaaacagaaataaacttcctgctcaaacaagcactcacgattgttgggactctgccatttacttacatgttagagaagtggaggtggatggtctttaaaggggaaattcccaaagaccagtggatgaaaaagtggtgggagatgaagcgagagatagttggggtggtggaacctgtgccccatgatgaaacatactgtgaccccgcatctctgttccatgtttctaatgattactcattcattcgatattacacaaggaccctttaccaattccagtttcaagaagcactttgtcaagcagctaaacatgaaggccctctgcacaaatgtgacatctcaaactctacagaagctggacagaaactgttcaatatgctgaggcttggaaaatcagaaccctggaccctagcattggaaaatgttgtaggagcaaagaacatgaatgtaaggccactgctcaactactttgagcccttatttacctggctgaaagaccagaacaagaattcttttgtgggatggagtaccgactggagtccatatgcagaccaaagcatcaaagtgaggataagcctaaaatcagctcttggagataaagcatatgaatggaacgacaatgaaatgtacctgttccgatcatctgttgcatatgctatgaggcagtactttttaaaagtaaaaaatcagatgattctttttggggaggaggatgtgcgagtggctaatttgaaaccaagaatctcctttaatttctttgtcactgcacctaaaaatgtgtctgatatcattcctagaactgaagttgaaaaggccatcaggatgtcccggagccgtatcaatgatgctttccgtctgaatgacaacagcctagagtttctggggatacagccaacacttggacctcctaaccagccccctgtttccatatggctgattgtttttggagttgtgatgggagtgatagtggttggcattgtcatcctgatcttcactgggatcagagatcggaagaagaaaaataaagcaagaagtggagaaaatccttatgcctccatcgatattagcaaaggagaaaataatccaggattccaaaacactgatgatgttcagacctccttttag

SEQ ID NO: 2 - DNA, 7518 bp, Betacoronavirus
atgttcgtcttcctggtcctgctgcctctggtctcctcacagtgcgtcaatttcacaactcggactcagctgccacctgcttatactaatagcttcaccagaggcgtgtactatcctgacaaggtgtttagaagctccgtgctgcactctacacaggatctgtttctgccattctttagcaacgtgacctggttccacgccatccacgtgagcggcaccaatggcacaaagcggttcgccaatcccgtgctgccttttaacgatggcgtgtacttcgcctctaccgagaagagcaacatcatcagaggctggatctttggcaccacactggactccaagacacagtctctgctgatcgtgaacaatgccaccaacgtggtcatcaaggtgtgcgagttccagttttgtaatgatcccttcctgggcgtgtactatcacaagaacaataagagctggatggagtccgagtttagagtgtattctagcgccaacaactgcacatttgagtacgtgagccagcctttcctgatggacctggagggcaagcagggcaatttcaagaacctgagggagttcgtgtttaagaatatcgacggctacttcaaaatctactctaagcacacccccatcaacctggtgcgcggcctgcctcagggcttcagcgccctggagcccctggtggatctgcctatcggcatcaacatcacccggtttcagacactgcacatcagctacctgacacccggcgactcctctagcggatggaccgccggcgctgccgcctactatgtgggctacctccagccccggaccttcctgctgaagtacaacgagaatggcaccatcacagacgcagtggattgcgccctggaccccctgagcgagacaaagtgtacactgaagtcctttaccgtggagaagggcatctatcagacatccaatttcagggtgcagccaaccgagtctatcgtgcgctttcctaatatcacaaacctgtgcccatttggcgaggtgttcaacgcaacccgcttcgccagcgtgtacgcctggaataggaagcggatcagcaactgcgtggccgactatagcgtgctgtacaactccgcctctttcagcacctttaagtgctatggcgtgtcccccacaaagctgaatgacctgtgctttaccaacgtctacgccgattctttcgtgatcaggggcgacgaggtgcgccagatcgcccccggccagacaggcaacatcgcagactacaattataagctgccagacgatttcaccggctgcgtgatcgcctggaacagcaacaatctggattccaaagtgggcggcaactacaattatctgtaccggctgtttagaaagagcaatctgaagcccttcgagagggacatctctacagaaatctaccaggccggcagcaccccttgcaatggcgtgaagggctttaactgttatttcccactccagtcctacggcttccagcccacatacggcgtgggctatcagccttaccgcgtggtggtgctgagctttgagctgctgcacgccccagcaacagtgtgcggccccaagaagtccaccaatctggtgaagaacaagtgcgtgaacttcaacttcaacggcctgaccggcacaggcgtgctgaccgagtccaacaagaagttcctgccatttcagcagttcggcagggacatcgcagataccacagacgccgtgcgcgacccacagaccctggagatcctggacatcacaccctgctctttcggcggcgtgagcgtgatcacacccggcaccaatacaagcaaccaggtggccgtgctgtatcagggcgtgaattgtaccgaggtgcccgtggctatccacgccgatcagctgaccccaacatggcgggtgtacagcaccggctccaacgtcttccagacaagagccggatgcctgatcggagcagagcacgtgaacaattcctatgagtgcgacatcccaatcggcgccggcatctgtgcctcttaccagacccagacaaactctcccagaagagcccggagcgtggcctcccagtctatcatcgcctataccatgtccctgggcgtggagaacagcgtggcctactctaacaatagcatcgccatcccaaccaacttcacaatctctgtgaccacagagatcctgcccgtgtccatgaccaagacatctgtggactgcacaatgtatatctgtggcgattctaccgagtgcagcaacctgctgctccagtacggcagcttttgtacccagctgaatagagccctgacaggcatcgccgtggagcaggataagaacacacaggaggtgttcgcccaggtgaagcaaatctacaagaccccccctatcaaggactttggcggcttcaatttttcccagatcctgcctgatccatccaagccttctaagcggagctttatcgaggacctgctgttcaacaaggtgaccctggccgatgccggcttcatcaagcagtatggcgattgcctgggcgacatcgcagccagggacctgatctgcgcccagaagtttaatggcctgaccgtgctgccacccctgctgacagatgagatgatcgcacagtacacaagcgccctgctggccggcaccatcacatccggatggaccttcggcgcaggagccgccctccagatcccctttgccatgcagatggcctataggttcaacggcatcggcgtgacccagaatgtgctgtacgagaaccagaagctgatcgccaatcagtttaactccgccatcggcaagatccaggacagcctgtcctctacagccagcgccctgggcaagctccaggatgtggtgaatcagaacgcccaggccctgaataccctggtgaagcagctgagcagcaacttcggcgccatctctagcgtgctgaatgacatcctgagccggctggacaaggtggaggcagaggtgcagatcgaccggctgatcaccggccggctccagagcctccagacctatgtgacacagcagctgatcagggccgccgagatcagggccagcgccaatctggcagcaaccaagatgtccgagtgcgtgctgggccagtctaagagagtggacttttgtggcaagggctatcacctgatgtccttccctcagtctgccccacacggcgtggtgtttctgcacgtgacctacgtgcccgcccaggagaagaacttcaccacagcccctgccatctgccacgatggcaaggcccactttccaagggagggcgtgttcgtgtccaacggcacccactggtttgtgacacagcgcaatttctacgagccccagatcatcaccacagacaacaccttcgtgagcggcaactgtgacgtggtcatcggcatcgtgaacaataccgtgtatgatccactccagcccgagctggacagctttaaggaggagctggataagtatttcaagaatcacacctcccctgacgtggatctgggcgacatcagcggcatcaatgcctccgtggtgaacatccagaaggagatcgaccgcctgaacgaggtggctaagaatctgaacgagagcctgatcgacctccaggagctgggcaagtatgagcagtacatcaagtggccctggtacatctggctgggcttcatcgccggcctgatcgccatcgtgatggtgaccatcatgctgtgctgtatgacatcctgctgttcttgcctgaagggctgctgtagctgtggctcctgctgtaagtaaatgttcgtcttcctggtcctgctgcctctggtctcctcacagtgcgtcaatttcacaactcggactcagctgccacctgcttatactaatagcttcaccagaggcgtgtactatcctgacaaggtgtttagaagctccgtgctgcactctacacaggatctgtttctgccattctttagcaacgtgacctggttccacgccatccacgtgagcggcaccaatggcacaaagcggttcgccaatcccgtgctgccttttaacgatggcgtgtacttcgcctctaccgagaagagcaacatcatcagaggctggatctttggcaccacactggactccaagacacagtctctgctgatcgtgaacaatgccaccaacgtggtcatcaaggtgtgcgagttccagttttgtaatgatcccttcctgggcgtgtactatcacaagaacaataagagctggatggagtccgagtttagagtgtattctagcgccaacaactgcacatttgagtacgtgagccagcctttcctgatggacctggagggcaagcagggcaatttcaagaacctgagggagttcgtgtttaagaatatcgacggctacttcaaaatctactctaagcacacccccatcaacctggtgcgcggcctgcctcagggcttcagcgccctggagcccctggtggatctgcctatcggcatcaacatcacccggtttcagacactgcacatcagctacctgacacccggcgactcctctagcggatggaccgccggcgctgccgcctactatgtgggctacctccagccccggaccttcctgctgaagtacaacgagaatggcaccatcacagacgcagtggattgcgccctggaccccctgagcgagacaaagtgtacactgaagtcctttaccgtggagaagggcatctatcagacatccaatttcagggtgcagccaaccgagtctatcgtgcgctttcctaatatcacaaacctgtgcccatttggcgaggtgttcaacgcaacccgcttcgccagcgtgtacgcctggaataggaagcggatcagcaactgcgtggccgactatagcgtgctgtacaactccgcctctttcagcacctttaagtgctatggcgtgtcccccacaaagctgaatgacctgtgctttaccaacgtctacgccgattctttcgtgatcaggggcgacgaggtgcgccagatcgcccccggccagacaggcaacatcgcagactacaattataagctgccagacgatttcaccggctgcgtgatcgcctggaacagcaacaatctggattccaaagtgggcggcaactacaattatctgtaccggctgtttagaaagagcaatctgaagcccttcgagagggacatctctacagaaatctaccaggccggcagcaccccttgcaatggcgtgaagggctttaactgttatttcccactccagtcctacggcttccagcccacatacggcgtgggctatcagccttaccgcgtggtggtgctgagctttgagctgctgcacgccccagcaacagtgtgcggccccaagaagtccaccaatctggtgaagaacaagtgcgtgaacttcaacttcaacggcctgaccggcacaggcgtgctgaccgagtccaacaagaagttcctgccatttcagcagttcggcagggacatcgcagataccacagacgccgtgcgcgacccacagaccctggagatcctggacatcacaccctgctctttcggcggcgtgagcgtgatcacacccggcaccaatacaagcaaccaggtggccgtgctgtatcagggcgtgaattgtaccgaggtgcccgtggctatccacgccgatcagctgaccccaacatggcgggtgtacagcaccggctccaacgtcttccagacaagagccggatgcctgatcggagcagagcacgtgaacaattcctatgagtgcgacatcccaatcggcgccggcatctgtgcctcttaccagacccagacaaactctcccagaagagcccggagcgtggcctcccagtctatcatcgcctataccatgtccctgggcgtggagaacagcgtggcctactctaacaatagcatcgccatcccaaccaacttcacaatctctgtgaccacagagatcctgcccgtgtccatgaccaagacatctgtggactgcacaatgtatatctgtggcgattctaccgagtgcagcaacctgctgctccagtacggcagcttttgtacccagctgaatagagccctgacaggcatcgccgtggagcaggataagaacacacaggaggtgttcgcccaggtgaagcaaatctacaagaccccccctatcaaggactttggcggcttcaatttttcccagatcctgcctgatccatccaagccttctaagcggagctttatcgaggacctgctgttcaacaaggtgaccctggccgatgccggcttcatcaagcagtatggcgattgcctgggcgacatcgcagccagggacctgatctgcgcccagaagtttaatggcctgaccgtgctgccacccctgctgacagatgagatgatcgcacagtacacaagcgccctgctggccggcaccatcacatccggatggaccttcggcgcaggagccgccctccagatcccctttgccatgcagatggcctataggttcaacggcatcggcgtgacccagaatgtgctgtacgagaaccagaagctgatcgccaatcagtttaactccgccatcggcaagatccaggacagcctgtcctctacagccagcgccctgggcaagctccaggatgtggtgaatcagaacgcccaggccctgaataccctggtgaagcagctgagcagcaacttcggcgccatctctagcgtgctgaatgacatcctgagccggctggacaaggtggaggcagaggtgcagatcgaccggctgatcaccggccggctccagagcctccagacctatgtgacacagcagctgatcagggccgccgagatcagggccagcgccaatctggcagcaaccaagatgtccgagtgcgtgctgggccagtctaagagagtggacttttgtggcaagggctatcacctgatgtccttccctcagtctgccccacacggcgtggtgtttctgcacgtgacctacgtgcccgcccaggagaagaacttcaccacagcccctgccatctgccacgatggcaaggcccactttccaagggagggcgtgttcgtgtccaacggcacccactggtttgtgacacagcgcaatttctacgagccccagatcatcaccacagacaacaccttcgtgagcggcaactgtgacgtggtcatcggcatcgtgaacaataccgtgtatgatccactccagcccgagctggacagctttaaggaggagctggataagtatttcaagaatcacacctcccctgacgtggatctgggcgacatcagcggcatcaatgcctccgtggtgaacatccagaaggagatcgaccgcctgaacgaggtggctaagaatctgaacgagagcctgatcgacctccaggagctgggcaagtatgagcagtacatcaagtggccctggtacatctggctgggcttcatcgccggcctgatcgccatcgtgatggtgaccatcatgctgtgctgtatgacatcctgctgttcttgcctgaagggctgctgtagctgtggctcctgctgtaagtaa

SEQ ID NO: 3 - DNA, 3855 bp, Betacoronavirus
atgtttgtcttcctggtcctgctgcctctggtctcgtctcagtgcgtgaacctgactactagaacccagctgcctcctgcctatactaactccttcacccgcggcgtgtactacccagacaaggtgttccgcagctccgtgctgcactccacccaggatctgttcctgcccttcttcagcaacgtgacctggttccacgccatccacgtgagcggcaccaatggcaccaagcggttcgacaatcccgtgctgccattcaacgatggcgtgtacttcgcctccaccgagaagagcaacatcatccgcggctggatcttcggcaccaccctggactccaagacccagagcctgctgatcgtgaacaatgccaccaacgtggtcatcaaggtgtgcgagttccagttctgcaatgatccattcctgggcgtgtactaccacaagaacaataagtcctggatggagagcgagttccgcgtgtacagctccgccaacaattgcaccttcgagtacgtgtcccagcccttcctgatggacctggagggcaagcagggcaatttcaagaacctgcgcgagttcgtgttcaagaatatcgatggctacttcaagatctactccaagcacacccccatcaacctggtgcgcgacctgccacagggcttcagcgccctggagccactggtggatctgccaatcggcatcaacatcaccaggttccagaccctgctggccctgcaccgcagctacctgaccccaggcgacagctccagcggatggaccgctggagctgctgcctactacgtgggctacctgcagccccgcaccttcctgctgaagtacaacgagaatggcaccatcaccgacgccgtggattgcgccctggatccactgtccgagacaaagtgcaccctgaagagcttcaccgtggagaagggcatctaccagacctccaatttccgcgtgcagccaaccgagagcatcgtgcgcttccccaatatcaccaacctgtgcccattcggcgaggtgttcaacgctaccaggttcgccagcgtgtacgcttggaatcgcaagcgcatctccaactgcgtggccgactacagcgtgctgtacaactccgccagcttctccaccttcaagtgctacggcgtgtcccccaccaagctgaatgatctgtgcttcaccaacgtgtacgccgatagcttcgtgatcaggggcgacgaggtgcgccagatcgctccaggacagaccggcaagatcgctgactacaattacaagctgcccgacgatttcaccggctgcgtgatcgcctggaactccaacaatctggatagcaaagtgggcggcaactacaattacctgtaccgcctgttccgcaagtccaatctgaagccattcgagcgcgacatctccaccgagatctaccaggctggaagcaccccatgcaatggagtggagggcttcaactgctacttccccctgcagagctacggcttccagccaaccaacggagtgggataccagccatacagggtggtggtgctgtccttcgagctgctgcacgctccagctaccgtgtgcggaccaaagaagagcaccaatctggtgaagaacaagtgcgtgaacttcaatttcaacggcctgaccggaaccggcgtgctgaccgagtccaacaagaagttcctgccattccagcagttcggaagggacatcgctgataccaccgacgccgtgcgcgacccacagaccctggagatcctggatatcaccccatgctccttcggcggcgtgagcgtgatcaccccaggaaccaataccagcaaccaggtggccgtgctgtaccaggacgtgaattgcaccgaggtgccagtggctatccacgctgatcagctgaccccaacctggcgcgtgtacagcaccggatccaacgtgttccagacccgcgccggatgcctgatcggagctgagcacgtgaacaattcctacgagtgcgacatcccaatcggagctggaatctgcgccagctaccagacccagaccaactccccaaggagggctcgcagcgtggccagccagtccatcatcgcctacaccatgtccctgggcgccgagaatagcgtggcctacagcaacaattccatcgccatcccaaccaacttcaccatctccgtgaccaccgagatcctgcccgtgtccatgaccaagaccagcgtggactgcaccatgtacatctgcggcgattccaccgagtgcagcaacctgctgctgcagtacggcagcttctgcacccagctgaatcgcgccctgaccggaatcgctgtggagcaggataagaacacccaggaggtgttcgcccaggtgaagcagatctacaagacccccccaatcaaggacttcggcggcttcaatttcagccagatcctgcccgatccaagcaagccctccaagcgcagcttcatcgaggacctgctgttcaacaaggtgaccctggccgatgccggcttcatcaagcagtacggcgattgcctgggcgacatcgctgcccgcgacctgatctgcgcccagaagttcaatggcctgaccgtgctgccaccactgctgaccgatgagatgatcgctcagtacacctccgccctgctggccggaaccatcaccagcggatggaccttcggcgctggagccgccctgcagatccccttcgccatgcagatggcctaccgcttcaacggcatcggcgtgacccagaatgtgctgtacgagaaccagaagctgatcgccaatcagttcaactccgccatcggcaagatccaggactccctgtccagcaccgccagcgccctgggcaagctgcaggatgtggtgaatcagaacgcccaggccctgaataccctggtgaagcagctgtccagcaacttcggcgccatctccagcgtgctgaatgatatcctgagccgcctggacaaggtggaggctgaggtgcagatcgataggctgatcaccggccgcctgcagtccctgcagacctacgtgacccagcagctgatcagggctgctgagatcagggccagcgccaatctggctgctaccaagatgtccgagtgcgtgctgggacagagcaagagggtggacttctgcggcaagggctaccacctgatgtccttcccacagagcgccccacacggagtggtgttcctgcacgtgacctacgtgccagcccaggagaagaacttcaccaccgctccagctatctgccacgatggcaaggctcacttcccacgcgagggcgtgttcgtgtccaacggcacccactggttcgtgacccagcgcaatttctacgagccccagatcatcaccaccgacaataccttcgtgagcggcaactgcgacgtggtcatcggaatcgtgaacaataccgtgtacgatcccctgcagccagagctggactccttcaaggaggagctggataagtacttcaagaatcacaccagccccgacgtggatctgggcgacatctccggcatcaatgccagcgtggtgaacatccagaaggagatcgaccgcctgaacgaggtggccaagaatctgaacgagtccctgatcgatctgcaggagctgggcaagtacgagcagtacatcaagtggccatggtacatctggctgggcttcatcgccggcctgatcgccatcgtgatggtgaccatcatgctgtgctgcatgacctcctgctgcagctgcctgaagggctgctgctcctgcggcagctgctgcaagttcgatgaggacgatagcgagcccgtgctgaagggcgtcaaactgcactatacaggctccaccgagacatcccaggtcgctcccgcttag

SEQ ID NO: 4 - DNA, 3855 bp, Artificial Sequence
  Spike protein D614G mutant
atgtttgtcttcctggtcctgctgcctctggtctcgtctcagtgcgtgaacctgactactagaacccagctgcctcctgcctatactaactccttcacccgcggcgtgtactacccagacaaggtgttccgcagctccgtgctgcactccacccaggatctgttcctgcccttcttcagcaacgtgacctggttccacgccatccacgtgagcggcaccaatggcaccaagcggttcgacaatcccgtgctgccattcaacgatggcgtgtacttcgcctccaccgagaagagcaacatcatccgcggctggatcttcggcaccaccctggactccaagacccagagcctgctgatcgtgaacaatgccaccaacgtggtcatcaaggtgtgcgagttccagttctgcaatgatccattcctgggcgtgtactaccacaagaacaataagtcctggatggagagcgagttccgcgtgtacagctccgccaacaattgcaccttcgagtacgtgtcccagcccttcctgatggacctggagggcaagcagggcaatttcaagaacctgcgcgagttcgtgttcaagaatatcgatggctacttcaagatctactccaagcacacccccatcaacctggtgcgcgacctgccacagggcttcagcgccctggagccactggtggatctgccaatcggcatcaacatcaccaggttccagaccctgctggccctgcaccgcagctacctgaccccaggcgacagctccagcggatggaccgctggagctgctgcctactacgtgggctacctgcagccccgcaccttcctgctgaagtacaacgagaatggcaccatcaccgacgccgtggattgcgccctggatccactgtccgagacaaagtgcaccctgaagagcttcaccgtggagaagggcatctaccagacctccaatttccgcgtgcagccaaccgagagcatcgtgcgcttccccaatatcaccaacctgtgcccattcggcgaggtgttcaacgctaccaggttcgccagcgtgtacgcttggaatcgcaagcgcatctccaactgcgtggccgactacagcgtgctgtacaactccgccagcttctccaccttcaagtgctacggcgtgtcccccaccaagctgaatgatctgtgcttcaccaacgtgtacgccgatagcttcgtgatcaggggcgacgaggtgcgccagatcgctccaggacagaccggcaagatcgctgactacaattacaagctgcccgacgatttcaccggctgcgtgatcgcctggaactccaacaatctggatagcaaagtgggcggcaactacaattacctgtaccgcctgttccgcaagtccaatctgaagccattcgagcgcgacatctccaccgagatctaccaggctggaagcaccccatgcaatggagtggagggcttcaactgctacttccccctgcagagctacggcttccagccaaccaacggagtgggataccagccatacagggtggtggtgctgtccttcgagctgctgcacgctccagctaccgtgtgcggaccaaagaagagcaccaatctggtgaagaacaagtgcgtgaacttcaatttcaacggcctgaccggaaccggcgtgctgaccgagtccaacaagaagttcctgccattccagcagttcggaagggacatcgctgataccaccgacgccgtgcgcgacccacagaccctggagatcctggatatcaccccatgctccttcggcggcgtgagcgtgatcaccccaggaaccaataccagcaaccaggtggccgtgctgtaccagggtgtgaattgcaccgaggtgccagtggctatccacgctgatcagctgaccccaacctggcgcgtgtacagcaccggatccaacgtgttccagacccgcgccggatgcctgatcggagctgagcacgtgaacaattcctacgagtgcgacatcccaatcggagctggaatctgcgccagctaccagacccagaccaactccccaaggagggctcgcagcgtggccagccagtccatcatcgcctacaccatgtccctgggcgccgagaatagcgtggcctacagcaacaattccatcgccatcccaaccaacttcaccatctccgtgaccaccgagatcctgcccgtgtccatgaccaagaccagcgtggactgcaccatgtacatctgcggcgattccaccgagtgcagcaacctgctgctgcagtacggcagcttctgcacccagctgaatcgcgccctgaccggaatcgctgtggagcaggataagaacacccaggaggtgttcgcccaggtgaagcagatctacaagacccccccaatcaaggacttcggcggcttcaatttcagccagatcctgcccgatccaagcaagccctccaagcgcagcttcatcgaggacctgctgttcaacaaggtgaccctggccgatgccggcttcatcaagcagtacggcgattgcctgggcgacatcgctgcccgcgacctgatctgcgcccagaagttcaatggcctgaccgtgctgccaccactgctgaccgatgagatgatcgctcagtacacctccgccctgctggccggaaccatcaccagcggatggaccttcggcgctggagccgccctgcagatccccttcgccatgcagatggcctaccgcttcaacggcatcggcgtgacccagaatgtgctgtacgagaaccagaagctgatcgccaatcagttcaactccgccatcggcaagatccaggactccctgtccagcaccgccagcgccctgggcaagctgcaggatgtggtgaatcagaacgcccaggccctgaataccctggtgaagcagctgtccagcaacttcggcgccatctccagcgtgctgaatgatatcctgagccgcctggacaaggtggaggctgaggtgcagatcgataggctgatcaccggccgcctgcagtccctgcagacctacgtgacccagcagctgatcagggctgctgagatcagggccagcgccaatctggctgctaccaagatgtccgagtgcgtgctgggacagagcaagagggtggacttctgcggcaagggctaccacctgatgtccttcccacagagcgccccacacggagtggtgttcctgcacgtgacctacgtgccagcccaggagaagaacttcaccaccgctccagctatctgccacgatggcaaggctcacttcccacgcgagggcgtgttcgtgtccaacggcacccactggttcgtgacccagcgcaatttctacgagccccagatcatcaccaccgacaataccttcgtgagcggcaactgcgacgtggtcatcggaatcgtgaacaataccgtgtacgatcccctgcagccagagctggactccttcaaggaggagctggataagtacttcaagaatcacaccagccccgacgtggatctgggcgacatctccggcatcaatgccagcgtggtgaacatccagaaggagatcgaccgcctgaacgaggtggccaagaatctgaacgagtccctgatcgatctgcaggagctgggcaagtacgagcagtacatcaagtggccatggtacatctggctgggcttcatcgccggcctgatcgccatcgtgatggtgaccatcatgctgtgctgcatgacctcctgctgcagctgcctgaagggctgctgctcctgcggcagctgctgcaagttcgatgaggacgatagcgagcccgtgctgaagggcgtcaaactgcactatacaggctccaccgagacatcccaggtcgctcccgcttag

SEQ ID NO: 5 - DNA, 3855 bp, Artificial Sequence
  Spike protein D614G/N501Y mutant
atgtttgtcttcctggtcctgctgcctctggtctcgtctcagtgcgtgaacctgactactagaacccagctgcctcctgcctatactaactccttcacccgcggcgtgtactacccagacaaggtgttccgcagctccgtgctgcactccacccaggatctgttcctgcccttcttcagcaacgtgacctggttccacgccatccacgtgagcggcaccaatggcaccaagcggttcgacaatcccgtgctgccattcaacgatggcgtgtacttcgcctccaccgagaagagcaacatcatccgcggctggatcttcggcaccaccctggactccaagacccagagcctgctgatcgtgaacaatgccaccaacgtggtcatcaaggtgtgcgagttccagttctgcaatgatccattcctgggcgtgtactaccacaagaacaataagtcctggatggagagcgagttccgcgtgtacagctccgccaacaattgcaccttcgagtacgtgtcccagcccttcctgatggacctggagggcaagcagggcaatttcaagaacctgcgcgagttcgtgttcaagaatatcgatggctacttcaagatctactccaagcacacccccatcaacctggtgcgcgacctgccacagggcttcagcgccctggagccactggtggatctgccaatcggcatcaacatcaccaggttccagaccctgctggccctgcaccgcagctacctgaccccaggcgacagctccagcggatggaccgctggagctgctgcctactacgtgggctacctgcagccccgcaccttcctgctgaagtacaacgagaatggcaccatcaccgacgccgtggattgcgccctggatccactgtccgagacaaagtgcaccctgaagagcttcaccgtggagaagggcatctaccagacctccaatttccgcgtgcagccaaccgagagcatcgtgcgcttccccaatatcaccaacctgtgcccattcggcgaggtgttcaacgctaccaggttcgccagcgtgtacgcttggaatcgcaagcgcatctccaactgcgtggccgactacagcgtgctgtacaactccgccagcttctccaccttcaagtgctacggcgtgtcccccaccaagctgaatgatctgtgcttcaccaacgtgtacgccgatagcttcgtgatcaggggcgacgaggtgcgccagatcgctccaggacagaccggcaagatcgctgactacaattacaagctgcccgacgatttcaccggctgcgtgatcgcctggaactccaacaatctggatagcaaagtgggcggcaactacaattacctgtaccgcctgttccgcaagtccaatctgaagccattcgagcgcgacatctccaccgagatctaccaggctggaagcaccccatgcaatggagtggagggcttcaactgctacttccccctgcagagctacggcttccagccaacctacggagtgggataccagccatacagggtggtggtgctgtccttcgagctgctgcacgctccagctaccgtgtgcggaccaaagaagagcaccaatctggtgaagaacaagtgcgtgaacttcaatttcaacggcctgaccggaaccggcgtgctgaccgagtccaacaagaagttcctgccattccagcagttcggaagggacatcgctgataccaccgacgccgtgcgcgacccacagaccctggagatcctggatatcaccccatgctccttcggcggcgtgagcgtgatcaccccaggaaccaataccagcaaccaggtggccgtgctgtaccagggtgtgaattgcaccgaggtgccagtggctatccacgctgatcagctgaccccaacctggcgcgtgtacagcaccggatccaacgtgttccagacccgcgccggatgcctgatcggagctgagcacgtgaacaattcctacgagtgcgacatcccaatcggagctggaatctgcgccagctaccagacccagaccaactccccaaggagggctcgcagcgtggccagccagtccatcatcgcctacaccatgtccctgggcgccgagaatagcgtggcctacagcaacaattccatcgccatcccaaccaacttcaccatctccgtgaccaccgagatcctgcccgtgtccatgaccaagaccagcgtggactgcaccatgtacatctgcggcgattccaccgagtgcagcaacctgctgctgcagtacggcagcttctgcacccagctgaatcgcgccctgaccggaatcgctgtggagcaggataagaacacccaggaggtgttcgcccaggtgaagcagatctacaagacccccccaatcaaggacttcggcggcttcaatttcagccagatcctgcccgatccaagcaagccctccaagcgcagcttcatcgaggacctgctgttcaacaaggtgaccctggccgatgccggcttcatcaagcagtacggcgattgcctgggcgacatcgctgcccgcgacctgatctgcgcccagaagttcaatggcctgaccgtgctgccaccactgctgaccgatgagatgatcgctcagtacacctccgccctgctggccggaaccatcaccagcggatggaccttcggcgctggagccgccctgcagatccccttcgccatgcagatggcctaccgcttcaacggcatcggcgtgacccagaatgtgctgtacgagaaccagaagctgatcgccaatcagttcaactccgccatcggcaagatccaggactccctgtccagcaccgccagcgccctgggcaagctgcaggatgtggtgaatcagaacgcccaggccctgaataccctggtgaagcagctgtccagcaacttcggcgccatctccagcgtgctgaatgatatcctgagccgcctggacaaggtggaggctgaggtgcagatcgataggctgatcaccggccgcctgcagtccctgcagacctacgtgacccagcagctgatcagggctgctgagatcagggccagcgccaatctggctgctaccaagatgtccgagtgcgtgctgggacagagcaagagggtggacttctgcggcaagggctaccacctgatgtccttcccacagagcgccccacacggagtggtgttcctgcacgtgacctacgtgccagcccaggagaagaacttcaccaccgctccagctatctgccacgatggcaaggctcacttcccacgcgagggcgtgttcgtgtccaacggcacccactggttcgtgacccagcgcaatttctacgagccccagatcatcaccaccgacaataccttcgtgagcggcaactgcgacgtggtcatcggaatcgtgaacaataccgtgtacgatcccctgcagccagagctggactccttcaaggaggagctggataagtacttcaagaatcacaccagccccgacgtggatctgggcgacatctccggcatcaatgccagcgtggtgaacatccagaaggagatcgaccgcctgaacgaggtggccaagaatctgaacgagtccctgatcgatctgcaggagctgggcaagtacgagcagtacatcaagtggccatggtacatctggctgggcttcatcgccggcctgatcgccatcgtgatggtgaccatcatgctgtgctgcatgacctcctgctgcagctgcctgaagggctgctgctcctgcggcagctgctgcaagttcgatgaggacgatagcgagcccgtgctgaagggcgtcaaactgcactatacaggctccaccgagacatcccaggtcgctcccgcttag

SEQ ID NO: 6 - DNA, 3855 bp, Artificial Sequence
  Spike protein D614G/N501Y/L18F/T20N/P26S mutant
atgtttgtcttcctggtcctgctgcctctggtctcgtctcagtgcgtgaacttcactaatagaacccagctgccttctgcctatactaactccttcacccgcggcgtgtactacccagacaaggtgttccgcagctccgtgctgcactccacccaggatctgttcctgcccttcttcagcaacgtgacctggttccacgccatccacgtgagcggcaccaatggcaccaagcggttcgacaatcccgtgctgccattcaacgatggcgtgtacttcgcctccaccgagaagagcaacatcatccgcggctggatcttcggcaccaccctggactccaagacccagagcctgctgatcgtgaacaatgccaccaacgtggtcatcaaggtgtgcgagttccagttctgcaatgatccattcctgggcgtgtactaccacaagaacaataagtcctggatggagagcgagttccgcgtgtacagctccgccaacaattgcaccttcgagtacgtgtcccagcccttcctgatggacctggagggcaagcagggcaatttcaagaacctgcgcgagttcgtgttcaagaatatcgatggctacttcaagatctactccaagcacacccccatcaacctggtgcgcgacctgccacagggcttcagcgccctggagccactggtggatctgccaatcggcatcaacatcaccaggttccagaccctgctggccctgcaccgcagctacctgaccccaggcgacagctccagcggatggaccgctggagctgctgcctactacgtgggctacctgcagccccgcaccttcctgctgaagtacaacgagaatggcaccatcaccgacgccgtggattgcgccctggatccactgtccgagacaaagtgcaccctgaagagcttcaccgtggagaagggcatctaccagacctccaatttccgcgtgcagccaaccgagagcatcgtgcgcttccccaatatcaccaacctgtgcccattcggcgaggtgttcaacgctaccaggttcgccagcgtgtacgcttggaatcgcaagcgcatctccaactgcgtggccgactacagcgtgctgtacaactccgccagcttctccaccttcaagtgctacggcgtgtcccccaccaagctgaatgatctgtgcttcaccaacgtgtacgccgatagcttcgtgatcaggggcgacgaggtgcgccagatcgctccaggacagaccggcaagatcgctgactacaattacaagctgcccgacgatttcaccggctgcgtgatcgcctggaactccaacaatctggatagcaaagtgggcggcaactacaattacctgtaccgcctgttccgcaagtccaatctgaagccattcgagcgcgacatctccaccgagatctaccaggctggaagcaccccatgcaatggagtggagggcttcaactgctacttccccctgcagagctacggcttccagccaacctacggagtgggataccagccatacagggtggtggtgctgtccttcgagctgctgcacgctccagctaccgtgtgcggaccaaagaagagcaccaatctggtgaagaacaagtgcgtgaacttcaatttcaacggcctgaccggaaccggcgtgctgaccgagtccaacaagaagttcctgccattccagcagttcggaagggacatcgctgataccaccgacgccgtgcgcgacccacagaccctggagatcctggatatcaccccatgctccttcggcggcgtgagcgtgatcaccccaggaaccaataccagcaaccaggtggccgtgctgtaccagggtgtgaattgcaccgaggtgccagtggctatccacgctgatcagctgaccccaacctggcgcgtgtacagcaccggatccaacgtgttccagacccgcgccggatgcctgatcggagctgagcacgtgaacaattcctacgagtgcgacatcccaatcggagctggaatctgcgccagctaccagacccagaccaactccccaaggagggctcgcagcgtggccagccagtccatcatcgcctacaccatgtccctgggcgccgagaatagcgtggcctacagcaacaattccatcgccatcccaaccaacttcaccatctccgtgaccaccgagatcctgcccgtgtccatgaccaagaccagcgtggactgcaccatgtacatctgcggcgattccaccgagtgcagcaacctgctgctgcagtacggcagcttctgcacccagctgaatcgcgccctgaccggaatcgctgtggagcaggataagaacacccaggaggtgttcgcccaggtgaagcagatctacaagacccccccaatcaaggacttcggcggcttcaatttcagccagatcctgcccgatccaagcaagccctccaagcgcagcttcatcgaggacctgctgttcaacaaggtgaccctggccgatgccggcttcatcaagcagtacggcgattgcctgggcgacatcgctgcccgcgacctgatctgcgcccagaagttcaatggcctgaccgtgctgccaccactgctgaccgatgagatgatcgctcagtacacctccgccctgctggccggaaccatcaccagcggatggaccttcggcgctggagccgccctgcagatccccttcgccatgcagatggcctaccgcttcaacggcatcggcgtgacccagaatgtgctgtacgagaaccagaagctgatcgccaatcagttcaactccgccatcggcaagatccaggactccctgtccagcaccgccagcgccctgggcaagctgcaggatgtggtgaatcagaacgcccaggccctgaataccctggtgaagcagctgtccagcaacttcggcgccatctccagcgtgctgaatgatatcctgagccgcctggacaaggtggaggctgaggtgcagatcgataggctgatcaccggccgcctgcagtccctgcagacctacgtgacccagcagctgatcagggctgctgagatcagggccagcgccaatctggctgctaccaagatgtccgagtgcgtgctgggacagagcaagagggtggacttctgcggcaagggctaccacctgatgtccttcccacagagcgccccacacggagtggtgttcctgcacgtgacctacgtgccagcccaggagaagaacttcaccaccgctccagctatctgccacgatggcaaggctcacttcccacgcgagggcgtgttcgtgtccaacggcacccactggttcgtgacccagcgcaatttctacgagccccagatcatcaccaccgacaataccttcgtgagcggcaactgcgacgtggtcatcggaatcgtgaacaataccgtgtacgatcccctgcagccagagctggactccttcaaggaggagctggataagtacttcaagaatcacaccagccccgacgtggatctgggcgacatctccggcatcaatgccagcgtggtgaacatccagaaggagatcgaccgcctgaacgaggtggccaagaatctgaacgagtccctgatcgatctgcaggagctgggcaagtacgagcagtacatcaagtggccatggtacatctggctgggcttcatcgccggcctgatcgccatcgtgatggtgaccatcatgctgtgctgcatgacctcctgctgcagctgcctgaagggctgctgctcctgcggcagctgctgcaagttcgatgaggacgatagcgagcccgtgctgaagggcgtcaaactgcactatacaggctccaccgagacatcccaggtcgctcccgcttag

SEQ ID NO: 7 - DNA, 3855 bp, Artificial Sequence
  Spike protein D614G/N501Y/T19R mutant
atgtttgtcttcctggtcctgctgcctctggtctcgtctcagtgcgtgaacctgagaactagaacccagctgcctcctgcctatactaactccttcacccgcggcgtgtactacccagacaaggtgttccgcagctccgtgctgcactccacccaggatctgttcctgcccttcttcagcaacgtgacctggttccacgccatccacgtgagcggcaccaatggcaccaagcggttcgacaatcccgtgctgccattcaacgatggcgtgtacttcgcctccaccgagaagagcaacatcatccgcggctggatcttcggcaccaccctggactccaagacccagagcctgctgatcgtgaacaatgccaccaacgtggtcatcaaggtgtgcgagttccagttctgcaatgatccattcctgggcgtgtactaccacaagaacaataagtcctggatggagagcgagttccgcgtgtacagctccgccaacaattgcaccttcgagtacgtgtcccagcccttcctgatggacctggagggcaagcagggcaatttcaagaacctgcgcgagttcgtgttcaagaatatcgatggctacttcaagatctactccaagcacacccccatcaacctggtgcgcgacctgccacagggcttcagcgccctggagccactggtggatctgccaatcggcatcaacatcaccaggttccagaccctgctggccctgcaccgcagctacctgaccccaggcgacagctccagcggatggaccgctggagctgctgcctactacgtgggctacctgcagccccgcaccttcctgctgaagtacaacgagaatggcaccatcaccgacgccgtggattgcgccctggatccactgtccgagacaaagtgcaccctgaagagcttcaccgtggagaagggcatctaccagacctccaatttccgcgtgcagccaaccgagagcatcgtgcgcttccccaatatcaccaacctgtgcccattcggcgaggtgttcaacgctaccaggttcgccagcgtgtacgcttggaatcgcaagcgcatctccaactgcgtggccgactacagcgtgctgtacaactccgccagcttctccaccttcaagtgctacggcgtgtcccccaccaagctgaatgatctgtgcttcaccaacgtgtacgccgatagcttcgtgatcaggggcgacgaggtgcgccagatcgctccaggacagaccggcaagatcgctgactacaattacaagctgcccgacgatttcaccggctgcgtgatcgcctggaactccaacaatctggatagcaaagtgggcggcaactacaattacctgtaccgcctgttccgcaagtccaatctgaagccattcgagcgcgacatctccaccgagatctaccaggctggaagcaccccatgcaatggagtggagggcttcaactgctacttccccctgcagagctacggcttccagccaacctacggagtgggataccagccatacagggtggtggtgctgtccttcgagctgctgcacgctccagctaccgtgtgcggaccaaagaagagcaccaatctggtgaagaacaagtgcgtgaacttcaatttcaacggcctgaccggaaccggcgtgctgaccgagtccaacaagaagttcctgccattccagcagttcggaagggacatcgctgataccaccgacgccgtgcgcgacccacagaccctggagatcctggatatcaccccatgctccttcggcggcgtgagcgtgatcaccccaggaaccaataccagcaaccaggtggccgtgctgtaccagggtgtgaattgcaccgaggtgccagtggctatccacgctgatcagctgaccccaacctggcgcgtgtacagcaccggatccaacgtgttccagacccgcgccggatgcctgatcggagctgagcacgtgaacaattcctacgagtgcgacatcccaatcggagctggaatctgcgccagctaccagacccagaccaactccccaaggagggctcgcagcgtggccagccagtccatcatcgcctacaccatgtccctgggcgccgagaatagcgtggcctacagcaacaattccatcgccatcccaaccaacttcaccatctccgtgaccaccgagatcctgcccgtgtccatgaccaagaccagcgtggactgcaccatgtacatctgcggcgattccaccgagtgcagcaacctgctgctgcagtacggcagcttctgcacccagctgaatcgcgccctgaccggaatcgctgtggagcaggataagaacacccaggaggtgttcgcccaggtgaagcagatctacaagacccccccaatcaaggacttcggcggcttcaatttcagccagatcctgcccgatccaagcaagccctccaagcgcagcttcatcgaggacctgctgttcaacaaggtgaccctggccgatgccggcttcatcaagcagtacggcgattgcctgggcgacatcgctgcccgcgacctgatctgcgcccagaagttcaatggcctgaccgtgctgccaccactgctgaccgatgagatgatcgctcagtacacctccgccctgctggccggaaccatcaccagcggatggaccttcggcgctggagccgccctgcagatccccttcgccatgcagatggcctaccgcttcaacggcatcggcgtgacccagaatgtgctgtacgagaaccagaagctgatcgccaatcagttcaactccgccatcggcaagatccaggactccctgtccagcaccgccagcgccctgggcaagctgcaggatgtggtgaatcagaacgcccaggccctgaataccctggtgaagcagctgtccagcaacttcggcgccatctccagcgtgctgaatgatatcctgagccgcctggacaaggtggaggctgaggtgcagatcgataggctgatcaccggccgcctgcagtccctgcagacctacgtgacccagcagctgatcagggctgctgagatcagggccagcgccaatctggctgctaccaagatgtccgagtgcgtgctgggacagagcaagagggtggacttctgcggcaagggctaccacctgatgtccttcccacagagcgccccacacggagtggtgttcctgcacgtgacctacgtgccagcccaggagaagaacttcaccaccgctccagctatctgccacgatggcaaggctcacttcccacgcgagggcgtgttcgtgtccaacggcacccactggttcgtgacccagcgcaatttctacgagccccagatcatcaccaccgacaataccttcgtgagcggcaactgcgacgtggtcatcggaatcgtgaacaataccgtgtacgatcccctgcagccagagctggactccttcaaggaggagctggataagtacttcaagaatcacaccagccccgacgtggatctgggcgacatctccggcatcaatgccagcgtggtgaacatccagaaggagatcgaccgcctgaacgaggtggccaagaatctgaacgagtccctgatcgatctgcaggagctgggcaagtacgagcagtacatcaagtggccatggtacatctggctgggcttcatcgccggcctgatcgccatcgtgatggtgaccatcatgctgtgctgcatgacctcctgctgcagctgcctgaagggctgctgctcctgcggcagctgctgcaagttcgatgaggacgatagcgagcccgtgctgaagggcgtcaaactgcactatacaggctccaccgagacatcccaggtcgctcccgcttag

SEQ ID NO: 8 - DNA, 3849 bp, Artificial Sequence
  Spike protein D614G/N501Y/HV 69-70deletion mutant
atgtttgtcttcctggtcctgctgcctctggtctcgtctcagtgcgtgaacctgactactagaacccagctgcctcctgcctatactaactccttcacccgcggcgtgtactacccagacaaggtgttccgcagctccgtgctgcactccacccaggatctgttcctgcccttcttcagcaacgtgacctggttccacgccatcagcggcaccaatggcaccaagcggttcgacaatcccgtgctgccattcaacgatggcgtgtacttcgcctccaccgagaagagcaacatcatccgcggctggatcttcggcaccaccctggactccaagacccagagcctgctgatcgtgaacaatgccaccaacgtggtcatcaaggtgtgcgagttccagttctgcaatgatccattcctgggcgtgtactaccacaagaacaataagtcctggatggagagcgagttccgcgtgtacagctccgccaacaattgcaccttcgagtacgtgtcccagcccttcctgatggacctggagggcaagcagggcaatttcaagaacctgcgcgagttcgtgttcaagaatatcgatggctacttcaagatctactccaagcacacccccatcaacctggtgcgcgacctgccacagggcttcagcgccctggagccactggtggatctgccaatcggcatcaacatcaccaggttccagaccctgctggccctgcaccgcagctacctgaccccaggcgacagctccagcggatggaccgctggagctgctgcctactacgtgggctacctgcagccccgcaccttcctgctgaagtacaacgagaatggcaccatcaccgacgccgtggattgcgccctggatccactgtccgagacaaagtgcaccctgaagagcttcaccgtggagaagggcatctaccagacctccaatttccgcgtgcagccaaccgagagcatcgtgcgcttccccaatatcaccaacctgtgcccattcggcgaggtgttcaacgctaccaggttcgccagcgtgtacgcttggaatcgcaagcgcatctccaactgcgtggccgactacagcgtgctgtacaactccgccagcttctccaccttcaagtgctacggcgtgtcccccaccaagctgaatgatctgtgcttcaccaacgtgtacgccgatagcttcgtgatcaggggcgacgaggtgcgccagatcgctccaggacagaccggcaagatcgctgactacaattacaagctgcccgacgatttcaccggctgcgtgatcgcctggaactccaacaatctggatagcaaagtgggcggcaactacaattacctgtaccgcctgttccgcaagtccaatctgaagccattcgagcgcgacatctccaccgagatctaccaggctggaagcaccccatgcaatggagtggagggcttcaactgctacttccccctgcagagctacggcttccagccaacctacggagtgggataccagccatacagggtggtggtgctgtccttcgagctgctgcacgctccagctaccgtgtgcggaccaaagaagagcaccaatctggtgaagaacaagtgcgtgaacttcaatttcaacggcctgaccggaaccggcgtgctgaccgagtccaacaagaagttcctgccattccagcagttcggaagggacatcgctgataccaccgacgccgtgcgcgacccacagaccctggagatcctggatatcaccccatgctccttcggcggcgtgagcgtgatcaccccaggaaccaataccagcaaccaggtggccgtgctgtaccagggtgtgaattgcaccgaggtgccagtggctatccacgctgatcagctgaccccaacctggcgcgtgtacagcaccggatccaacgtgttccagacccgcgccggatgcctgatcggagctgagcacgtgaacaattcctacgagtgcgacatcccaatcggagctggaatctgcgccagctaccagacccagaccaactccccaaggagggctcgcagcgtggccagccagtccatcatcgcctacaccatgtccctgggcgccgagaatagcgtggcctacagcaacaattccatcgccatcccaaccaacttcaccatctccgtgaccaccgagatcctgcccgtgtccatgaccaagaccagcgtggactgcaccatgtacatctgcggcgattccaccgagtgcagcaacctgctgctgcagtacggcagcttctgcacccagctgaatcgcgccctgaccggaatcgctgtggagcaggataagaacacccaggaggtgttcgcccaggtgaagcagatctacaagacccccccaatcaaggacttcggcggcttcaatttcagccagatcctgcccgatccaagcaagccctccaagcgcagcttcatcgaggacctgctgttcaacaaggtgaccctggccgatgccggcttcatcaagcagtacggcgattgcctgggcgacatcgctgcccgcgacctgatctgcgcccagaagttcaatggcctgaccgtgctgccaccactgctgaccgatgagatgatcgctcagtacacctccgccctgctggccggaaccatcaccagcggatggaccttcggcgctggagccgccctgcagatccccttcgccatgcagatggcctaccgcttcaacggcatcggcgtgacccagaatgtgctgtacgagaaccagaagctgatcgccaatcagttcaactccgccatcggcaagatccaggactccctgtccagcaccgccagcgccctgggcaagctgcaggatgtggtgaatcagaacgcccaggccctgaataccctggtgaagcagctgtccagcaacttcggcgccatctccagcgtgctgaatgatatcctgagccgcctggacaaggtggaggctgaggtgcagatcgataggctgatcaccggccgcctgcagtccctgcagacctacgtgacccagcagctgatcagggctgctgagatcagggccagcgccaatctggctgctaccaagatgtccgagtgcgtgctgggacagagcaagagggtggacttctgcggcaagggctaccacctgatgtccttcccacagagcgccccacacggagtggtgttcctgcacgtgacctacgtgccagcccaggagaagaacttcaccaccgctccagctatctgccacgatggcaaggctcacttcccacgcgagggcgtgttcgtgtccaacggcacccactggttcgtgacccagcgcaatttctacgagccccagatcatcaccaccgacaataccttcgtgagcggcaactgcgacgtggtcatcggaatcgtgaacaataccgtgtacgatcccctgcagccagagctggactccttcaaggaggagctggataagtacttcaagaatcacaccagccccgacgtggatctgggcgacatctccggcatcaatgccagcgtggtgaacatccagaaggagatcgaccgcctgaacgaggtggccaagaatctgaacgagtccctgatcgatctgcaggagctgggcaagtacgagcagtacatcaagtggccatggtacatctggctgggcttcatcgccggcctgatcgccatcgtgatggtgaccatcatgctgtgctgcatgacctcctgctgcagctgcctgaagggctgctgctcctgcggcagctgctgcaagttcgatgaggacgatagcgagcccgtgctgaagggcgtcaaactgcactatacaggctccaccgagacatcccaggtcgctcccgcttag

SEQ ID NO: 9 - DNA, 411 bp, Streptomyces griseus
atggccaagcctttgtctcaagaagaatccaccctcattgaaagagcaacggctacaatcaacagcatccccatctctgaagactacagcgtcgccagcgcagctctctctagcgacggccgcatcttcactggtgtcaatgtatatcattttactgggggaccttgtgcagaactcgtggtgctgggcactgctgctgctgcggcagctggcaacctgacttgtatcgtcgcgatcggaaatgagaacaggggcatcttgagcccctgcggacggtgccgacaggtgcttctcgatctgcatcctgggatcaaagccatagtgaaggacagtgatggacagccgacggcagttgggattcgtgaattgctgccctctggttatgtgtgggagggcctgcagctgcagtag

(19) United States
(12) Patent Application Publication
(10) Pub. No.: US 2023/0321138 A1
(43) Pub. Date: Oct. 12, 2023

(54) METHODS OF PREVENTING OR TREATING CORONAVIRUS INFECTION AND REDUCING CORONAVIRUS INFECTION RATE IN VITRO WITH ANTIVIRAL COMPOSITION COMPRISING POLY(3,4-ETHYLENEDIOXYTHIOPHENE):POLY(STYRENE SULFONATE)

(71) Applicant: NATIONAL YANG MING CHIAO TUNG UNIVERSITY, Taipei City (TW)

(72) Inventor: Ming-Han TSAI, Taipei City (TW)

(21) Appl. No.: 17/839,991

(22) Filed: Jun. 14, 2022

(30) Foreign Application Priority Data
Apr. 11, 2022 (TW) 111113701

(51) Int. Cl. A61K 31/795; A61P 31/14

(52) U.S. Cl. CPC A61K 31/795; A61P 31/14

(57) ABSTRACT

The present invention provides methods of preventing or treating coronavirus infection and reducing coronavirus infection rate in vitro with antiviral composition comprising poly(3,4-ethylenedioxythiophene):poly(styrene sulfonate) (PEDOT:PSS), wherein the PEDOT:PSS is selected from PEDOT:PSS aqueous solutions in various formulas. Specifically, the molar ratio of PEDOT to PSS in the PEDOT:PSS aqueous solution in the antiviral composition may be 1:1.5 to 1:6 and the resistivity of the PEDOT:PSS aqueous solution in the antiviral composition may be 100 Ω/sq to 5×10⁷ Ω/sq. The antiviral composition is used to inhibit the binding between the spike protein of the coronavirus and the host cells with angiotensin-converting enzyme 2 (ACE2) on surface. The coronavirus may be SARS-CoV-2.

### CROSS-REFERENCE TO RELATED APPLICATIONS

This application claims the benefit of priority of Taiwan Application Number TW111113701, filed on Apr. 11, 2022, which is herein incorporated by reference in its entirety.

### TECHNICAL FIELD

The present invention is related to methods of preventing or treating coronavirus infection and reducing coronavirus infection rate in vitro with antiviral composition comprising poly(3,4-ethylenedioxythiophene):poly(styrene sulfonate) (PEDOT:PSS). Specifically, the coronavirus may be SARS-CoV-2. The PEDOT:PSS is selected from PEDOT:PSS aqueous solutions in various formulas.

### BACKGROUND

Covid-19, which is caused by severe acute respiratory syndrome coronavirus 2 (SARS-CoV-2), has been a pandemic disease since 2019. SARS-CoV-2 is a coronavirus which infects host cells through utilizing the spike protein on the surface of the virus to bind ACE2 (angiotensin-converting enzyme 2) expressed on the host cells surface. Numerous vaccines for Covid-19 have been available. However, because coronavirus is an RNA virus which has high mutation rate, new mutants develop quickly. Thus, vaccines derived from the original virus strain may fail to prevent transmission of numerous known SARS-CoV-2 mutant strains with immune escape property (William T. Harvey et al., Nature Reviews Microbiology 19, pages 409-424, 2021). Therefore, facing quick development of new SARS-CoV-2 mutants, we need a broad-spectrum antiviral substance to defend against SARS-CoV-2. Nevertheless, most of the broad-spectrum antiviral substances such as hypochlorous acid are toxic for human. Accordingly, there has been no broad-spectrum antiviral substance that can be used to reduce coronavirus infection rate both in vivo and in vitro.

Poly(3,4-ethylenedioxythiophene):poly(styrene sulfonate) (PEDOT:PSS) is a conductive macromolecule. Thanks to the conductivity and high biocompatibility of PEDOT:PSS, PEDOT:PSS has been applied in many biomedical materials. In addition, it had also been reported that PEDOT:PSS/agarose hydrogels are able to block bacterial growth (Youngsang Ko et al., Carbohydr Polym 2019, 203, 26-34.). However, there is no description, suggestion or teaching about whether PEDOT:PSS can be used in pharmaceutical composition or antiviral composition for coronavirus in the prior art.

### SUMMARY

Given the technical problem described above, the present invention is intended to provide a method of reducing infection rate of different coronavirus mutants both in vivo and in vitro by using PEDOT:PSS aqueous solution as a broad-spectrum antiviral substance.

The present invention provides a method of preventing or treating coronavirus infection in a subject, comprising a step of administering a therapeutically effective amount of antiviral composition comprising poly(3,4-ethylenedioxythiophene):poly(styrene sulfonate) (PEDOT:PSS), wherein the

PEDOT:PSS is selected from PEDOT:PSS aqueous solutions in various formulas. The present invention also provides a method of reducing coronavirus infection rate in vitro by using antiviral composition comprising PEDOT:PSS, wherein the PEDOT:PSS is selected from PEDOT:PSS aqueous solutions in various formulas.

In one aspect, the molar ratio of PEDOT to PSS in the PEDOT:PSS aqueous solution in the antiviral composition may be 1:1.5 to 1:6.

In one aspect, the resistivity of the PEDOT:PSS aqueous solution in the antiviral composition may be 100 Ω/sq to 5×10⁷ Ω/sq.

In one aspect, the antiviral composition is used to inhibit binding of spike protein of the coronavirus to host cells with ACE2 (angiotensin-converting enzyme 2) on surface.

In one aspect, the coronavirus may be SARS-CoV-2.

In one aspect, the antiviral composition may comprise other antiviral drugs or other antiviral chemicals.

The present invention provides a method of preventing or treating coronavirus infection with antiviral composition comprising PEDOT:PSS, wherein the PEDOT:PSS is selected from PEDOT:PSS aqueous solutions in various formulas which can impede the binding of the coronavirus to host cells. In addition, the broad-spectrum antiviral composition is able to reduce infection rate of different coronavirus mutants for host cells in the face of high mutation rate of coronavirus. The antiviral composition comprising PEDOT:PSS aqueous solution can also be used in reducing infection rate of different coronavirus mutants for host cells in vitro.

### BRIEF DESCRIPTION OF THE DRAWINGS

FIG. 1 demonstrates the impact of PEDOT:PSS aqueous solution on cell viability evaluated in Example 3 by illustrating the cell viability of HEK293T/hACE2 cells treated with various dilutions of PEDOT:PSS aqueous solution.

FIG. 2A, FIG. 2B, FIG. 2C and FIG. 2D demonstrate the effect of PEDOT:PSS aqueous solution on reducing coronavirus infection rate evaluated in Example 4. FIG. 2A and FIG. 2B show the infection rate analyzed by fluorescence microscope and flow cytometry respectively. FIG. 2C and FIG. 2D show the infection rate of higher concentration (10-fold original concentration) of Spike pseudovirus analyzed by fluorescence microscope and flow cytometry respectively in Example 4.

FIG. 3A and FIG. 3B demonstrate the effect of pretreating Spike pseudovirus with PEDOT:PSS aqueous solution evaluated in Example 5 and show the infection rate analyzed by fluorescence microscope and flow cytometry respectively.

FIG. 4A, FIG. 4B, FIG. 4C and FIG. 4D demonstrate the effect of pretreating HEK293T/hACE2 cells with PEDOT:PSS aqueous solution evaluated in Example 5. FIG. 4A and FIG. 4B show the infection rate analyzed by fluorescence microscope and flow cytometry respectively. FIG. 4C and FIG. 4D show the infection rate analyzed by fluorescence microscope and flow cytometry respectively when HEK293T/hACE2 cells were pretreated for different periods in Example 5.

FIG. 5A and FIG. 5B demonstrate the effect of PEDOT:PSS aqueous solution on inhibiting the binding of Spike pseudovirus to HEK293T/hACE2 cells evaluated in Example 6 and show the amount of Spike pseudoviruses bound to HEK293T/hACE2 cells analyzed by fluorescence microscope and flow cytometry respectively.

FIG. 6 demonstrates the impact of PEDOT:PSS aqueous solutions in formulas 1-5 on cell viability evaluated in Example 7 by illustrating the cell viability of HEK293T/hACE2 cells treated with various dilutions of PEDOT:PSS aqueous solutions in formulas 1-5.

FIG. 7A and FIG. 7B demonstrate the effects of PEDOT:PSS aqueous solutions in formulas 1-5 on reducing coronavirus infection rate evaluated in Example 7 and show the infection rate analyzed by fluorescence microscope and flow cytometry respectively.

FIG. 8A and FIG. 8B demonstrate the effect of pretreating Spike pseudovirus with PEDOT:PSS aqueous solutions in various formulas evaluated in Example 7 and show the infection rate analyzed by fluorescence microscope and flow cytometry respectively.

FIG. 9A and FIG. 9B demonstrate the effect of PEDOT:PSS aqueous solution on reducing infection rate of various coronavirus mutants evaluated in Example 8 and show the infection rate analyzed by fluorescence microscope and flow cytometry respectively.

### DETAILED DESCRIPTION

The embodiments disclosed below are intended to describe the purpose and the effect of the present invention, not to limit the scope of the present invention.

The molar ratio of PEDOT to PSS in PEDOT:PSS aqueous solution used in the following exemplary Examples 1-6 and Example 8 is 1:6 with 1.3%-1.7% solid content in water (Clevios™ PEDOT:PSS A14083 from Heraeus Electronic Materials), which is also the PEDOT:PSS aqueous solution in the original formula in Example 7. PEDOT:PSS aqueous solutions in formulas 1-5 used in the following exemplary Example 7 are PEDOT:PSS aqueous solutions with different mixing ratio and resistivity (DAILY POLYMER Corporation, Taiwan) as shown in Table 1.

### EXAMPLES

The embodiments disclosed below are intended to evaluate the effect of PEDOT:PSS aqueous solution of the present invention on reducing coronavirus infection rate, wherein all the experimental data in each example are shown as the means ± standard deviations (SDs). Statistics analysis is conducted with Student's t test. In figures, asterisk, *, represents a statistically significant difference. The length of scale bars in microscope images represents 200 μm.

### Example 1: Establishment of Cell Model Expressing ACE2 (Angiotensin-Converting Enzyme 2)

Human ACE2 (hACE2) gene (SEQ ID NO: 1) and blasticidin resistance gene (SEQ ID NO: 9) were transduced into human HEK293T cells derived from human embryonic kidney cells expressing SV40 T antigen by lentivirus. HEK293T cells stably expressing human ACE2 protein (hereafter HEK293T/hACE2 cells) were obtained by blasticidin selection at concentration of 10 μg/ml. All cells used in the following examples were cultured with RPMI medium (Invitrogen) supplemented with 10% fetal calf serum (FBS, HyClone) in a humidified cell culture incubator at 37° C.

### Example 2: Establishment of Pseudovirus Model Expressing Spike Protein

pCMV-d8.91 plasmids, pLAS2-GFP lentiviral plasmids and plasmids encoding spike gene were transfected into HEK293T cells at the ratio of 6.25:6.5:1.1 by polyethylenimine (PEI). The supernatants were collected at the 60th hour post transfection and passed through 0.45 μm filters. The filtered liquid comprised pseudovirus expressing spike protein (hereafter also Spike pseudovirus). The pLAS2-GFP was prepared by inserting sequence encoding GFP gene after the sequence encoding CMV promoter in the pLAS2 lentiviral plasmid. The plasmids encoding spike gene comprised sequence encoding SARS-CoV-2 spike gene. Pseudovirus prepared in this way was based on lentivirus and expressed both of SARS-CoV-2 spike on its envelope and GFP. Therefore, the cells successfully infected by Spike pseudovirus were GFP-positive and could be analyzed by fluorescence microscope or flow cytometry.

The SARS-CoV-2 Spike proteins of Spike pseudovirus used in the following Examples 3-7 contained D614G and N501Y mutation with a cytoplasmic tail deletion of 13 amino acids encoded by nucleotides sequence as SEQ ID NO: 2. The corresponding sequences encoding wildtype SARS-CoV-2 Spike protein or those with specific mutation of Spike pseudoviruses used in the following Example 8 are: SEQ ID NO: 3 for wildtype, SEQ ID NO: 4 for D614G mutant, SEQ ID NO: 5 for D614G/N501Y mutant, SEQ ID NO: 6 for D614G/N501Y/L18F/T20N/P26S mutant, SEQ ID NO: 7 for D614G/N501Y/T19R and SEQ ID NO: 8 for D614G/N501Y/HV 69-70 deletion mutant respectively.

### Example 3: Evaluation of Impact of PEDOT:PSS Aqueous Solution on Cell Viability

To evaluate of the impact of PEDOT:PSS aqueous solution on cell viability, HEK293/hACE2 cells were seeded onto a 96-well culture plate with density 3×10³ cells per well and incubated overnight. Then, cells were treated with culture medium containing the different indicated dilutions of PEDOT:PSS aqueous solution for 48 hours. Cells in the control group were treated with culture medium without PEDOT:PSS aqueous solution. Cell viability of cells treated with PEDOT:PSS aqueous solution was evaluated by methylthiazol tetrazolium assay (MTT assay).

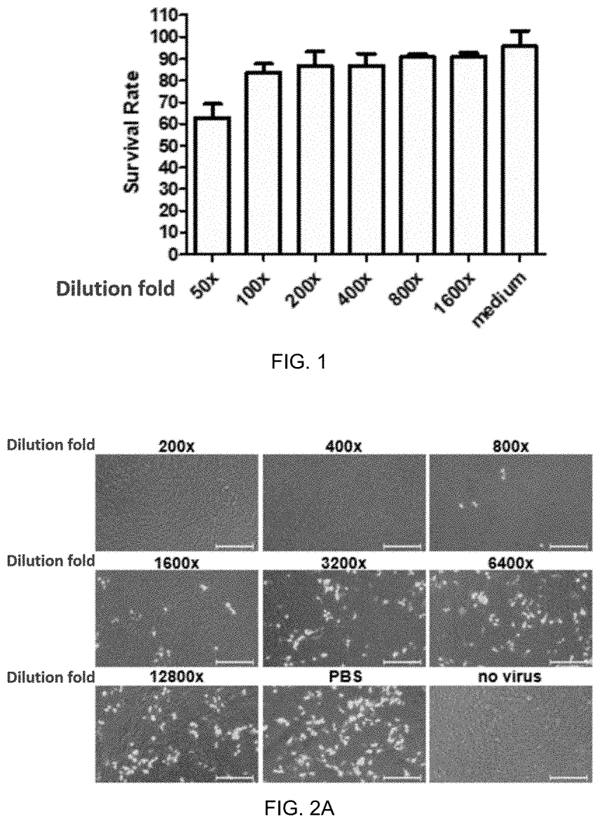

As shown in FIG. 1, except high concentration of PEDOT:PSS aqueous solution (50-fold dilution), cell viability of HEK293/hACE2 cells treated with PEDOT:PSS aqueous solution in other groups was not affected in 48 hours post-treatment.

### Example 4: Evaluation of Effect of PEDOT:PSS Aqueous Solution on Reducing Coronavirus Infection Rate

This example is intended to evaluate whether PEDOT:PSS aqueous solution can reduce the infection rate of pseudovirus with SARS-CoV-2 Spike proteins. The established Spike pseudovirus model as described above also expressed green fluorescent protein (GFP), so the cells successfully infected by Spike pseudovirus were GFP-positive and could be analyzed by fluorescence microscope or flow cytometry. HEK293/hACE2 cells were cultured in culture medium containing both the indicated dilutions of PEDOT:PSS aqueous solution and Spike pseudoviruses, wherein the infectious titer of Spike pseudovirus could infect 30% to 70% of HEK293T/hACE2 cells. After incubated for 48 hours, infection rate was analyzed by fluorescence microscope and flow cytometry. In the positive control group, culture medium containing phosphate buffered saline (PBS) and Spike pseudoviruses was used. In the negative control group, culture medium without Spike pseudovirus was used.

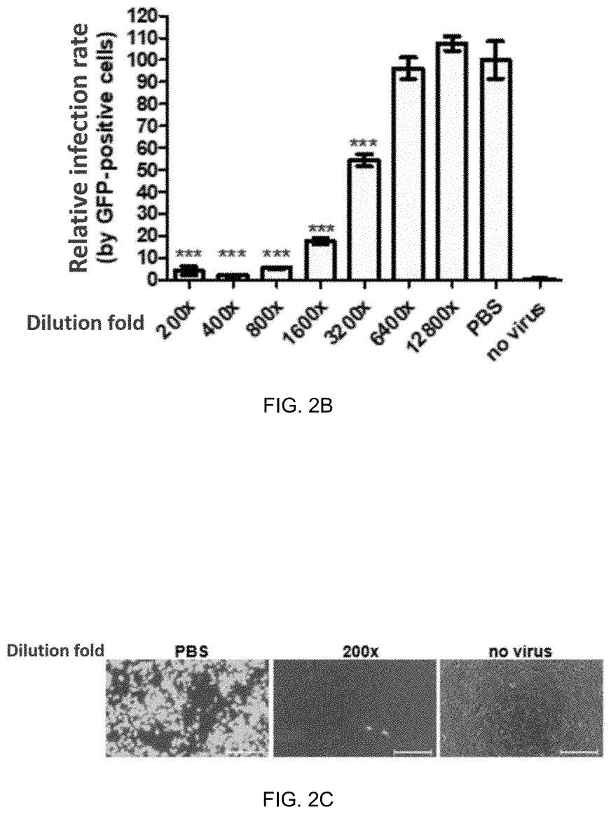

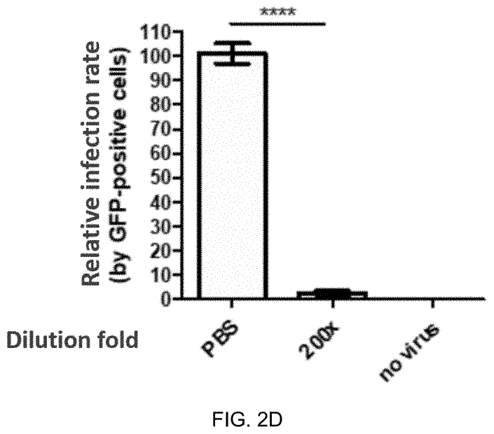

As shown in FIG. 2A, the amount of GFP-positive cells was reduced—that is to say, there were fewer infected cells—in the groups treated with lower dilutions (higher concentrations) of PEDOT:PSS aqueous solution. As shown in FIG. 2B, we barely observed any infected cell when the dilution of PEDOT:PSS aqueous solution was lower than 400-fold, and the relative inhibition rates detected by flow cytometry could reach nearly 100%. The results represented that treatment with PEDOT:PSS aqueous solution could significantly reduce the proportion of successfully infected HEK293T/hACE2 cells during Spike pseudovirus infection. For example, treatment with 3200-fold dilution could reduce the relative infection rate to about 50%. Also, the effect of inhibiting infection became more significant with the increase of dose. It was demonstrated that PEDOT:PSS aqueous solution indeed had the ability to inhibit SARS-CoV-2 Spike pseudovirus from infecting HEK293T/hACE2 cells.

It was further tested with higher concentration (10-fold original concentration) of Spike pseudovirus. As shown in FIG. 2C and FIG. 2D, PEDOT:PSS aqueous solution could still greatly reduce the proportion of HEK293T/hACE2 cells successfully infected by SARS-CoV-2 Spike pseudovirus. These experimental data showed that PEDOT:PSS aqueous solution could reduce coronavirus infection rate.

### Example 5: Evaluation of Effect of PEDOT:PSS Aqueous Solution on Preventing Spike Pseudovirus From Infecting HEK293T/hACE2 cells

To evaluate the effect of PEDOT PSS aqueous solution on preventing Spike pseudovirus infection, the effect of PEDOT:PSS aqueous solution on lowering infection ability of SARS-CoV-2 Spike pseudovirus was evaluated first. Spike pseudoviruses were pretreated with 200-fold dilution of PEDOT:PSS aqueous solution for 10 minutes at 37° C. Then, the supernatant containing PEDOT:PSS aqueous solution was entirely removed by gradient centrifugation. After centrifugation, the Spike pseudoviruses were further resuspended in PBS and used to carry out infection test as previously described in Example 4. In the control group, Spike pseudoviruses were pretreated with PBS only.

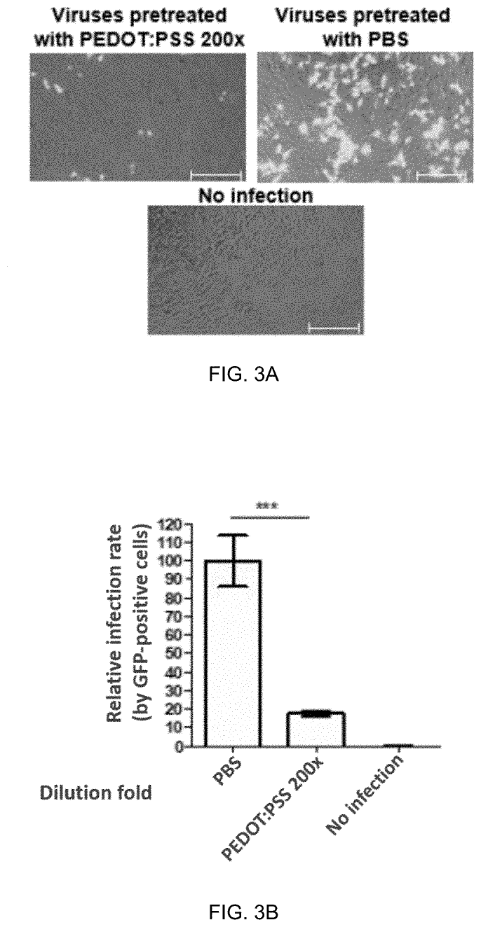

As shown in FIG. 3A and FIG. 3B, Spike pseudoviruses pretreated with PEDOT:PSS aqueous solution infected much fewer cells and lost about 80% of the infectivity compared with the positive control group.

In addition, the effect of preventing infection by pretreating HEK293T/hACE2 cells with PEDOT:PSS aqueous solution was also evaluated. HEK293T/hACE2 cells were pretreated with 200-fold dilution of PEDOT:PSS aqueous solution for 48 hours. The culture medium containing PEDOT:PSS aqueous solution was entirely removed and replaced with the culture medium containing Spike pseudoviruses to carry out infection test. HEK293T/hACE2 cells were pretreated with PBS only in the control group.

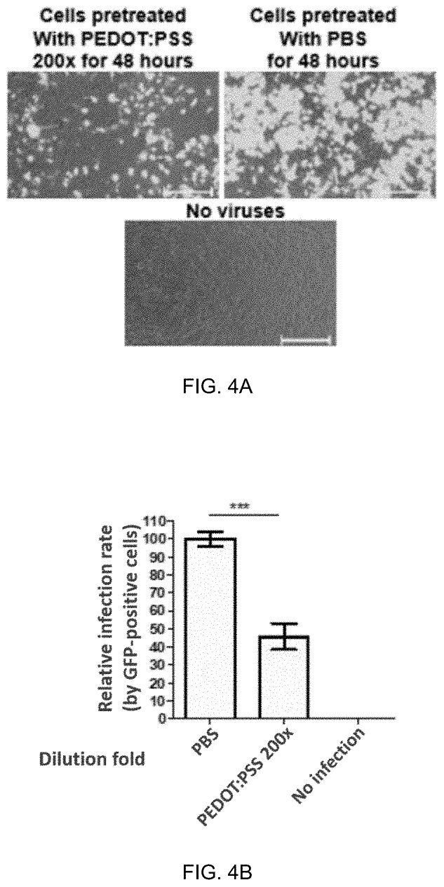

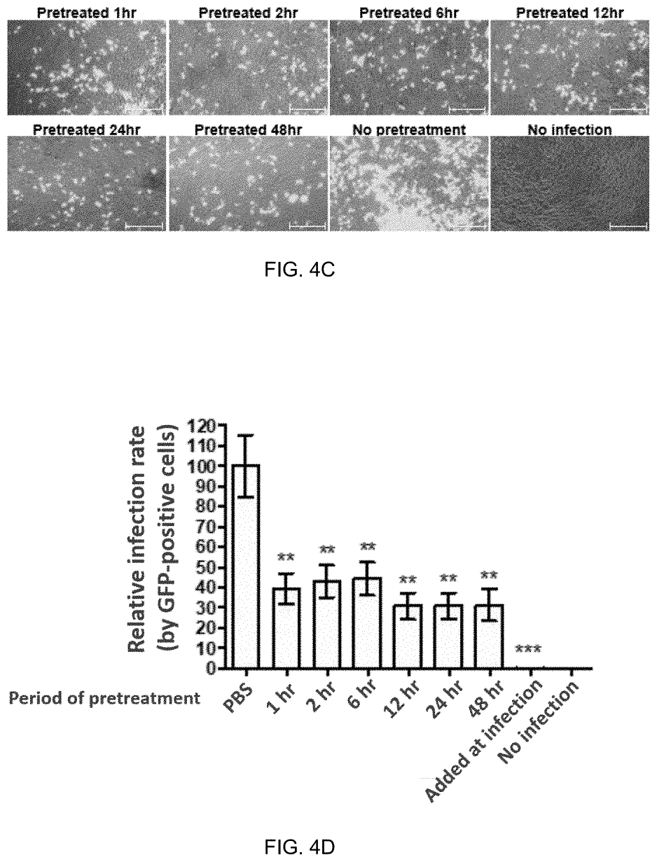

As shown in FIG. 4A and FIG. 4B, HEK293T/hACE2 cells pretreated with PEDOT:PSS aqueous solution for 48 hours showed about 50% reduction of infection rate in comparison with the control group.

The effect of preventing infection by pretreating HEK293T/hACE2 cells with PEDOT:PSS aqueous solution for different periods was further tested. As shown in FIG. 4C and FIG. 4D, the test results showed that the infection rate of HEK293T/hACE2 cells pretreated with PEDOT:PSS aqueous solution for only 1 hour was reduced by almost 50% in comparison with the control group.

The results described above showed that treating SARS-CoV-2 Spike pseudovirus with PEDOT:PSS aqueous solution reduced the ability to infect host cells of viruses (infection rate). Treating potential host cells with PEDOT:PSS aqueous solution could also prevent infection of Spike pseudovirus to a certain extent.

### Example 6: Evaluation of Effect of PEDOT:PSS Aqueous Solution on Inhibiting the Binding of Spike Pseudovirus to HEK293T/hACE2 Cells

HEK293T/hACE2 cells were pretreated with PEDOT:PSS aqueous solution or PBS for 1 hour and then subjected to a viral binding assay. Spike pseudoviruses and HEK293T/hACE2 cells were co-cultured under constant rolling for 1 hour at 4° C. The cells were collected by centrifugation, washed with PBS to remove Spike pseudoviruses which did not bind to cells. In another group, cells without or with PEDOT:PSS aqueous solution pre-treatment were treated with Spike pseudoviruses and PEDOT PSS aqueous solution simultaneously. Next, cells were dropped on glass slides. Immunofluorescence staining assay was performed with the rabbit polyclonal antibody as primary antibody to detect the Spike pseudoviruses bound to HEK293T/hACE2 cells. The samples analyzed by fluorescence microscope used Cyanine dye 3 conjugated (Cy3-conjugated) secondary antibody in immunofluorescence staining assay. The samples analyzed by flow cytometry used phycoerythrin conjugated (PE-conjugated) secondary antibody in immunofluorescence staining assay. Cells in the negative control group analyzed by flow cytometry were not stained by immunofluorescence staining assay.

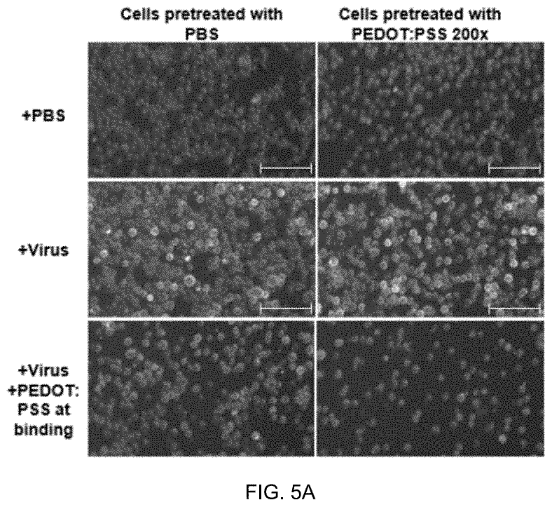

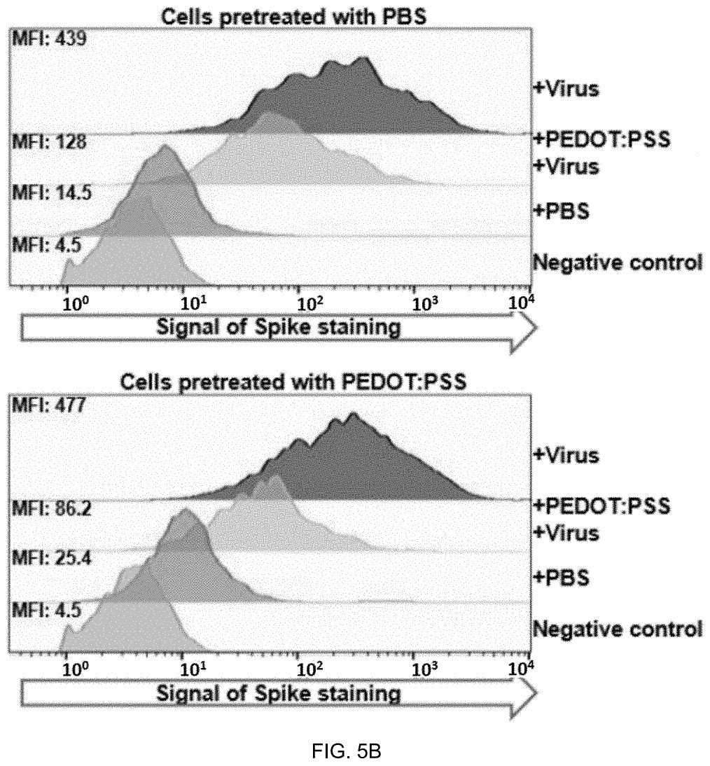

As shown in FIG. 5A and FIG. 5B, the effect of pretreating HEK293T/hACE2 cells with PEDOT:PSS aqueous solution was negligible. The amount of the SARS-CoV-2 Spike pseudovirus bound to HEK293T/hACE2 cells was significantly reduced when the cells were treated with Spike pseudovirus and PEDOT:PSS aqueous solution simultaneously. The results suggested that PEDOT:PSS aqueous solution reduced coronavirus infection rate by inhibiting spike protein of coronavirus from binding to host cells with ACE2 on surface.

### Example 7: Evaluation of Effects of PEDOT:PSS Aqueous Solutions in Various Formulas on Reducing Coronavirus Infection Rate

PEDOT:PSS is a polymer that can be formulated with different molar ratios of poly(3,4-ethylenedioxythiophene) (PEDOT) and poly(styrene sulfonate) (PSS).

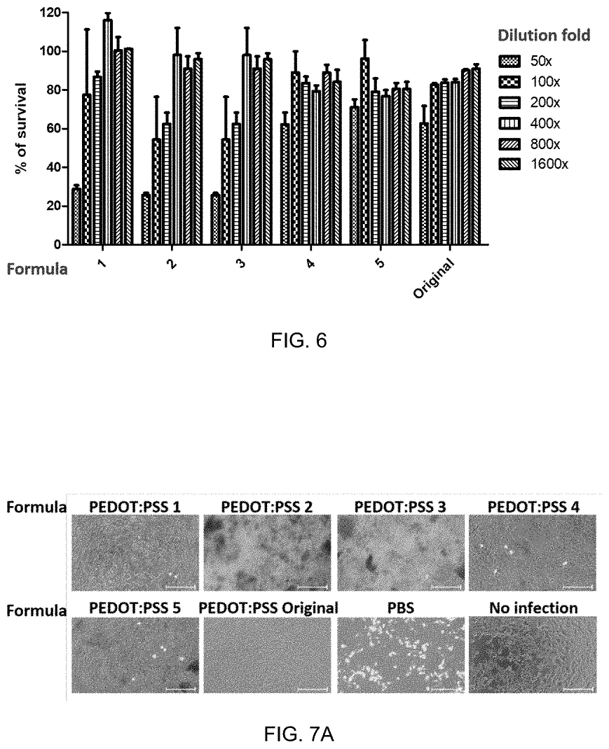

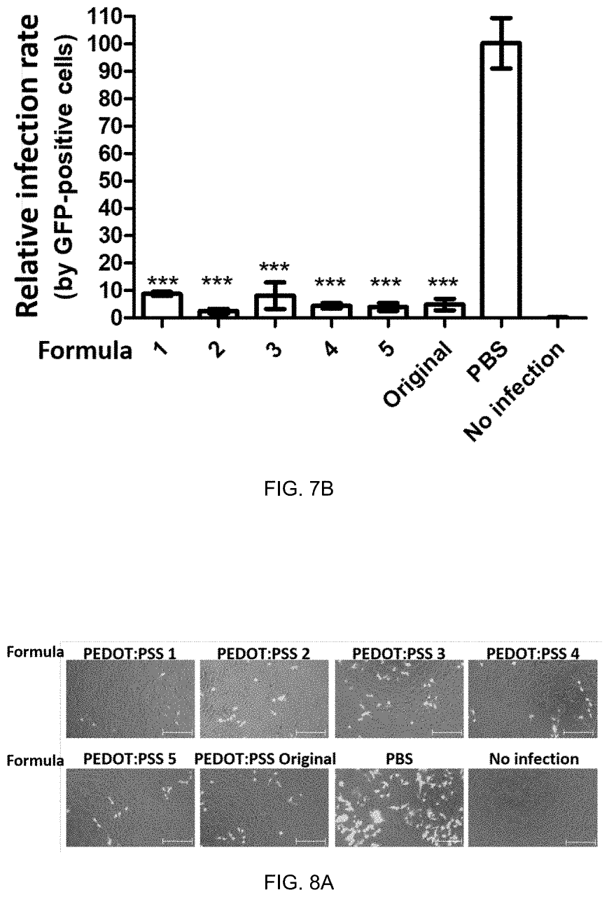

PEDOT:PSS with different conductivity could also be obtained by using different strategies for polymerization. To evaluate whether the effects of PEDOT:PSS aqueous solutions in various formulas on reducing coronavirus infection rate differ, the impacts of PEDOT:PSS aqueous solutions in formulas 1-5 with different composition ratios and different resistivities on cell viability were first tested by the method previously described in Example 3. The results are shown in FIG. 6. Subsequently, through the method previously described in Example 4, the effects of 200-fold dilutions of PEDOT:PSS aqueous solutions in formulas 1-5 on coronavirus infection rate were tested. The results are shown in FIG. 7A and FIG. 7B. The resistivity of each aqueous solution was measured by routine method in the technical field to which the present invention pertains after the PEDOT:PSS formed films. The composition ratios and resistivities of the PEDOT:PSS aqueous solutions in formulas 1-5 described above are shown in Table 1. The PEDOT:PSS aqueous solution in original formula in this example and Table 1 is the PEDOT:PSS aqueous solution used in Examples 1-6 described above. This PEDOT:PSS aqueous solution in original formula was also used in the Example 8 described below.

TABLE 1

| PEDOT:PSS | | | | | | | |
|---|---|---|---|---|---|---|---|
| aqueous solution | | | | | | | Original |
| formula | 1 | 2 | 3 | 4 | 5 | 6 | formula |
| Molar ratio of | 1:5 | 1:2.5 | 1:1.5 | 1:2.5 | 1:1.6 | 1:1.6 | 1:6 |
| PEDOT to PSS | | | | | | | |
| Resistivity (Ω/sq) | 10⁷ | 10⁷ | 10⁷ | 10² | 10² | 10² | 5 × 10⁶- |
| | | | | | | | 5 × 10⁷ |

As shown in FIG. 6, like PEDOT:PSS aqueous solution in original formula, except the groups treated with higher concentration (50-fold and 100-fold dilutions), cell viability of HEK293T/hACE2 cells in other groups treated with lower concentration of PEDOT:PSS aqueous solutions in formulas 1-5 was not significantly affected in 48 hours post-treatment.

As shown in FIG. 7A and FIG. 7B, like PEDOT:PSS aqueous solution in original formula, there were barely any GFP-positive cells in the groups treated with 200-fold dilution of PEDOT:PSS aqueous solutions in formulas 1-5 in comparison with the control group. The results represented that supplementing with PEDOT:PSS aqueous solutions in formulas 1-5 during infection could all significantly reduce the proportion of HEK293T/hACE2 cells successfully infected by Spike pseudovirus , which means that PEDOT:PSS aqueous solutions in various formulas could all inhibit the ability of SARS-CoV-2 Spike pseudovirus to infect HEK293T/hACE2 cells.

To evaluate the effect of pretreating Spike pseudovirus with PEDOT:PSS aqueous solutions in various formulas, the test previously described in Example 5 was performed with 200-fold dilution of PEDOT:PSS aqueous solutions in formulas 1-5.

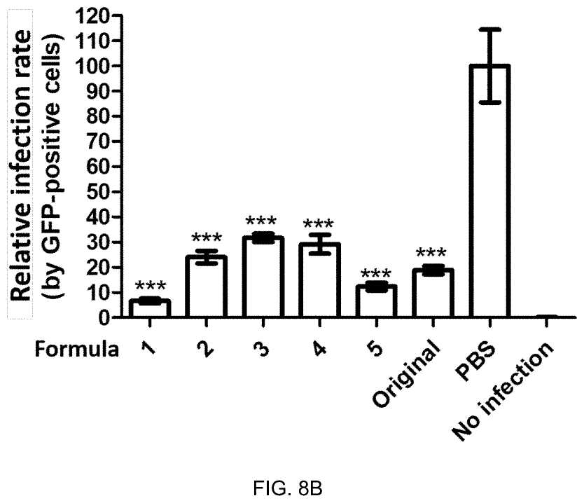

As shown in FIG. 8A and FIG. 8B, pretreating Spike pseudovirus with 200-fold dilution of PEDOT:PSS aqueous solutions in formulas 1-5 could reduce infection rate significantly in comparison with the control group.

The results described above suggested that PEDOT:PSS aqueous solutions in various formulas with different composition ratios or other properties all had potential to reduce infection rate of coronavirus with spike protein for host cells.

### Example 8: Evaluation of Effect of PEDOT:PSS Aqueous Solution as Broad-Spectrum Antiviral Substance on Reducing Infection Rate of Various Coronavirus Mutants

This example is intended to evaluate whether PEDOT:PSS aqueous solution can be used as broad-spectrum antiviral substance for reducing infection rate of virus mutants with various mutations. The test previously described in Example 4 was performed with Spike pseudovirus with various mutations on spike protein as described above and 200-fold dilution of PEDOT:PSS aqueous solution in this example. As previously described, mutations on spike protein for test included D614G mutation, D614G/N501Y mutation, D614G/N501Y/L18F/T20N/P26S mutation, D614G/N501Y/T19R mutation and D614G/N501Y/HV 69-70 deletion mutation. It has been disclosed that these mutations increase the infection rate of virus in the prior art.

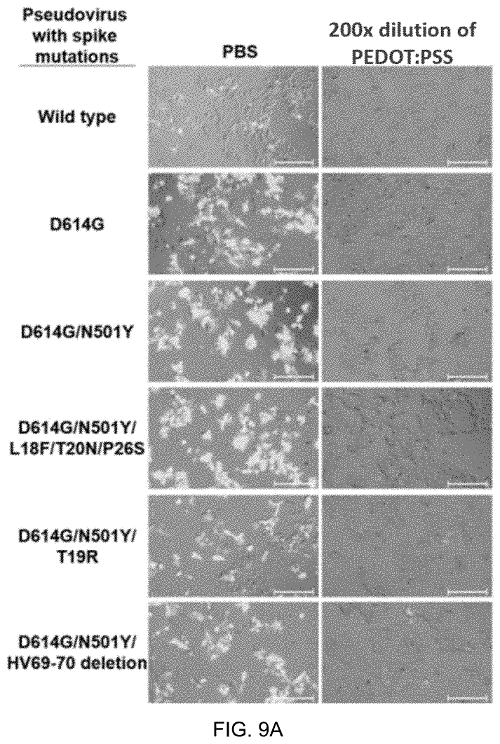

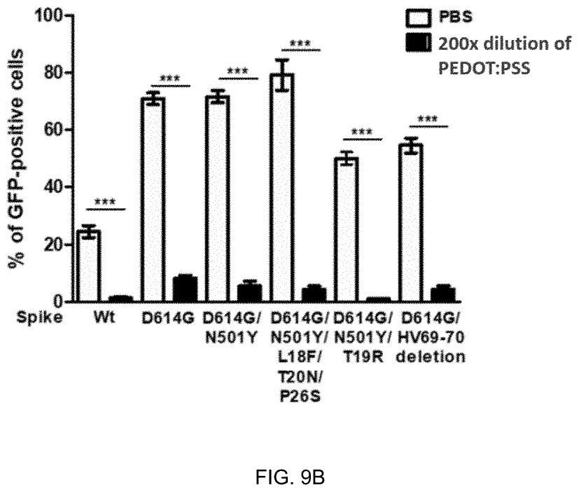

As shown in FIG. 9A, in the groups with spike protein with various mutations, significantly fewer GFP-positive cells could be found in the groups incubated with PEDOT:PSS aqueous solution in comparison with the control group. As shown in FIG. 9B, PEDOT:PSS aqueous solution could inhibit infection rate of various Spike pseudovirus mutants efficiently. The infection rates were all reduced to lower than nearly 10% after treatment and this inhibition effect was almost independent of mutations. The results suggested that PEDOT:PSS aqueous solution can be used as broad-spectrum antiviral substance for reducing infection rate of various coronavirus mutants efficiently.

This application contains references to amino acid sequences and/or nucleic acid sequences which have been submitted concurrently herewith as the sequence listing text file entitled “000005us_SequenceListing.TXT”, file size 44 kilobytes (KB), created on 9 May 2022. The aforementioned sequence listing is hereby incorporated by reference in its entirety pursuant to 37 C.F.R. § 1.52(e)(5).

What is claimed is:

1. A method of preventing or treating coronavirus infection in a subject, comprising a step of administering a therapeutically effective amount of antiviral composition comprising poly(3,4-ethylenedioxythiophene):poly(styrene sulfonate) (PEDOT:PSS), wherein the poly(3,4-ethylenedioxythiophene):poly(styrene sulfonate) (PEDOT:PSS) is selected from PEDOT:PSS aqueous solutions in various formulas.

2. A method of reducing coronavirus infection rate in vitro by using antiviral composition comprising poly(3,4-ethylenedioxythiophene):poly(styrene sulfonate) (PEDOT:PSS), wherein the poly(3,4-ethylenedioxythiophene):poly(styrene sulfonate) (PEDOT:PSS) is selected from PEDOT:PSS aqueous solutions in various formulas.

3. The method according to claim 1, wherein the molar ratio of PEDOT to PSS in the PEDOT:PSS aqueous solution in the antiviral composition is 1:1.5 to 1:6.

4. The method according to claim 2, wherein the molar ratio of PEDOT to PSS in the PEDOT:PSS aqueous solution in the antiviral composition is 1:1.5 to 1:6.

5. The method according to claim 1, wherein the resistivity of the PEDOT:PSS aqueous solution in the antiviral composition is 100 Ω/sq to 5×10⁷ Ω/sq.

6. The method according to claim 2, wherein the resistivity of the PEDOT:PSS aqueous solution in the antiviral composition is 100 Ω/sq to 5×10⁷ Ω/sq.

7. The method according to claim 1, wherein the antiviral composition is used to inhibit binding of spike protein of the coronavirus to host cells with ACE2 (angiotensin-converting enzyme 2) on surface.

8. The method according to claim 2, wherein the antiviral composition is used to inhibit binding of spike protein of the coronavirus to host cells with ACE2 (angiotensin-converting enzyme 2) on surface.

9. The method according to claim 1, wherein the coronavirus is SARS-CoV-2.

10. The method according to claim 2, wherein the coronavirus is SARS-CoV-2.

11. The method according to claim 1, wherein the antiviral composition comprises other antiviral drugs or other antiviral chemicals.

12. The method according to claim 2, wherein the antiviral composition comprises other antiviral drugs or other antiviral chemicals.
